(19) United States
(12) Patent Application Publication
(10) Pub. No.: US 2019/0192811 A1
(43) Pub. Date: Jun. 27, 2019

(54) Medical Tube with Add-on Endoscopic Capabilities

(71) Applicant: OTU Medical Inc., a California corporation, Fremont, CA (US)

(72) Inventors: XiBo WEI, Hayward, CA (US); GePing Liu, San Jose, CA (US); XiYi Wei, NingBo (CN)

(73) Assignee: OTU Medical Inc., a California corporation, Fremont, CA (US)

(21) Appl. No.: 15/854,009

(22) Filed: Dec. 26, 2017

(51) Int. Cl. A61M 25/00; A61M 16/04; A61J 15/00; A61B 1/06; A61B 6/00; A61B 1/05; A61B 1/00; A61B 1/12

(52) U.S. Cl. CPC A61M 25/003; A61M 16/0463; A61J 15/0003; A61B 1/0676; A61B 1/0684; A61B 6/44; A61M 25/10; A61M 25/0045; A61B 1/00114; A61B 1/00117; A61B 1/00029; A61B 1/126; A61M 2210/1085; A61B 1/05

(57) ABSTRACT

The present invention proposes a method of transforming a traditional, single-use medical tube to a novel one with add-on endoscopic capabilities. A tiny, transparent shell is added at the distal end of the medical tube for containing lighting sources inside, and the lighting source can be any small-size LED, OLED, or μLED device. Moreover, the tube has one additional lumen for installing power wires of the lighting sources, and another isolated lumen for inserting a flexible endoscope (video/image camera system) to observe targets inside human body. For minimizing outer diameter and cost, this endoscope does not include any built-in lighting source. While in use, the endoscope is first inserted into the medical tube through its specific lumen, and the lighting source at the tip is on. Secondly, the whole medical tube is safely inserted into human body for diagnosis or treatment with the help of endoscopic videos. In general, the medical tube is disposable, and the endoscope can be reused after proper sterilization procedures.

### RELATED APPLICATIONS

This application is a continuation-in-part (CIP) application of prior U.S. application Ser. No. 15/790,914 filed on 2017 Oct. 23 and prior U.S. application Ser. No. 15/823,582 filed on 2017 Nov. 28. The present application seeks the same priorities as that of the related applications in part. The entire contents of each of which are incorporated herein by reference.

### BACKGROUND OF THE INVENTION

### 1. Field of the Invention

The present invention belongs to a field of biomedical instrumentation. More specifically the present invention belongs to a field of medical tubing.

### 2. Description of the Related Art

Medical tubes are used to help patients. A catheter is generally necessary when someone can't empty his/her bladder. A tracheal tube is inserted into the trachea to establish and maintain a patient's airway and to ensure the adequate exchange of oxygen and carbon dioxide. Nasogastric tube is used when one desires to keep a stomach of the patient continuously and completely empty/evacuated of swallowed air, swallowed saliva, gastric secretions or fresh blood. It can also be used as a way of controlling or promoting feeding.

### BRIEF SUMMARY OF THE PRESENT INVENTION

The present invention is a medical tube with add-on endoscopic capabilities. In various embodiments, image sensors are built in on the tube, and are removable after use. In various embodiments, one or more video/image camera(s) as add-on devices is/are inserted into a medical tube such as a catheter through a reserved lumen.

A first embodiment of the present invention has a lighting source installed inside a transparent shell of a catheter with a preinstalled video/image camera system. The catheter has four lumens and four funnels. The lighting source and the video/image camera system can be removed from the catheter after use.

A second embodiment of the present invention has a catheter and an individual video/image camera system that can enter the catheter through a reserved lumen if needed. The catheter has four lumens and four funnels. The lighting source and the video/image camera system can be removed from the catheter after use.

A third embodiment of the present invention has a lighting source installed inside the transparent shell of the catheter with a preinstalled video/image camera system. The catheter has a double-layer balloon, five lumens and five funnels. The lighting source and the video/image camera system can move freely inside the lumens. The build-in video/image camera system can reach both the transparent shell and the balloon, same as the lighting source.

A fourth embodiment of the present invention has a catheter with preinstalled video/image camera system and two lighting sources with one at the transparent shell and one at balloon are not removable. The video/image camera system can move freely in the lumen, and can be reused. The lighting sources are not removable in this embodiment.

A fifth embodiment of the present invention has a catheter and an individual video/image camera system. The catheter has two lighting sources with one at the transparent shell and one at balloon are not removable. The video/image camera system can move freely in the lumen, and can be reused. The lighting sources are not removable in this embodiment.

A sixth embodiment of the present invention has a catheter with a preinstalled video/image camera system. The catheter has the lighting source installed inside the transparent shell of the catheter, and the distal end of the catheter is made of a transparent material. The catheter has a double-layer balloon, five lumens and five funnels. The lighting source and the video/image camera system can move freely inside the lumens. The build-in video/image camera system can reach both the transparent shell and the balloon, same as the lighting source.

A seventh embodiment of the present invention has a catheter and an individual video/image camera system. The catheter has a balloon, and a lighting source inside the transparent shell. The video/image camera system can move freely in the lumen, and can be reused.

An eighth embodiment of the present invention is a tracheal tube with endoscopic capabilities. A ninth embodiment of the present invention is a nasogastric tube with endoscopic capabilities.

The present invention includes a video/image camera system without requirement of a lighting source, and a method of using the medical tube with add-on endoscopic capabilities.

### BRIEF DESCRIPTION OF THE DRAWINGS

FIG. 1 illustrates the first embodiment of the present invention.

FIG. 2A illustrates a structure of the video/image camera system.

FIG. 2B is a top view of the video/image camera system.

FIG. 3 illustrates details of the distal end and an adjacent part of the catheter.

FIG. 4 illustrates two embodiments of configuration of a second lumen and the lighting source(s).

FIG. 5 illustrates a cross-section view of the catheter.

FIG. 6 illustrates the second embodiment of the present invention.

FIG. 7 illustrates the third embodiment of the present invention

FIG. 8 illustrates a portion of the present invention when the balloon is inflated.

FIG. 9 illustrates a cross-section view of the catheter.

FIG. 10 illustrates the fourth embodiment of the present invention.

FIG. 11 illustrates a portion of the present invention when the balloon is inflated.

FIG. 12 illustrates the fifth embodiment of the urinary catheter with endoscopic capabilities.

FIG. 13 illustrates the sixth embodiment the urinary catheter with endoscopic capabilities.

FIG. 14 illustrates the eighth embodiment of a tracheal tube with endoscopic capabilities.

FIG. 15 illustrates the ninth embodiment, a nasogastric tube with endoscopic capabilities.

FIG. 16 illustrates an embodiment of the video/image camera system with a wireless battery and a wireless charger.

### DETAILED DESCRIPTION OF THE INVENTION

The following description with reference to exemplary and illustration drawings of the present invention will be further described in detail, but the present illustration is not intended to limit the embodiment of the present invention, any similar structure of the present invention and similar changes should be included in the scope of the present invention.

The present invention is a medical tube with add-on endoscopic capabilities. The present invention includes but is not limited to catheter, gastric tube, nasogastric tube, feeding tube, rectal tube, and tracheal tube, with endoscopic capabilities, feeding tubes, tracheal tubes and so on that can be inserted into human body.

The first seven embodiments take the catheter with add-on endoscopic capabilities as example, and the features of the first seven embodiments can be applied to other medical tubes.

The urinary catheter with add-on endoscopic capabilities has a generally tubular-shaped housing with a distal end (121), a catheter shaft (120) and a proximal end (122). Lighting sources (190&510) can be installed in either the distal end (121) or the balloon (110) depending on the design requirements. Multiple lumens are fabricated in the generally tubular-shaped housing, and multiple funnels can be made at the proximal end (122).

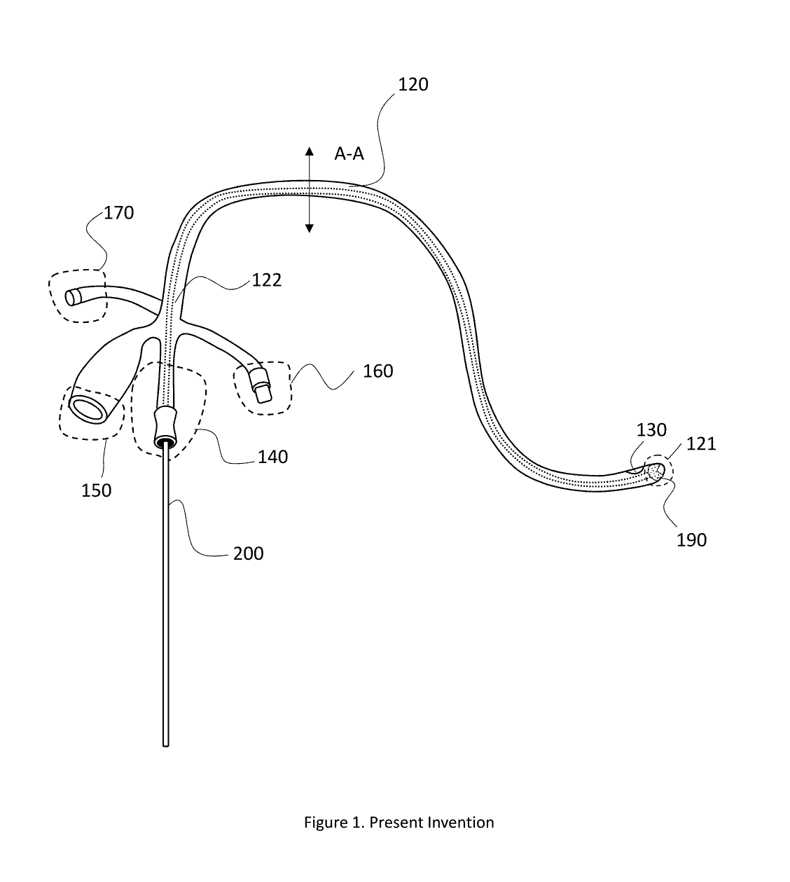

The first embodiment of the present invention is shown in FIG. 1. The catheter with a preinstalled video/image camera system (200) is general tubular-shaped. The catheter shaft (120) has one end connected to the distal end (121), and one end connected to the proximal end (122) where four funnels (140-170) joint. The lighting source (190) is installed inside the transparent shell (192) of the catheter.

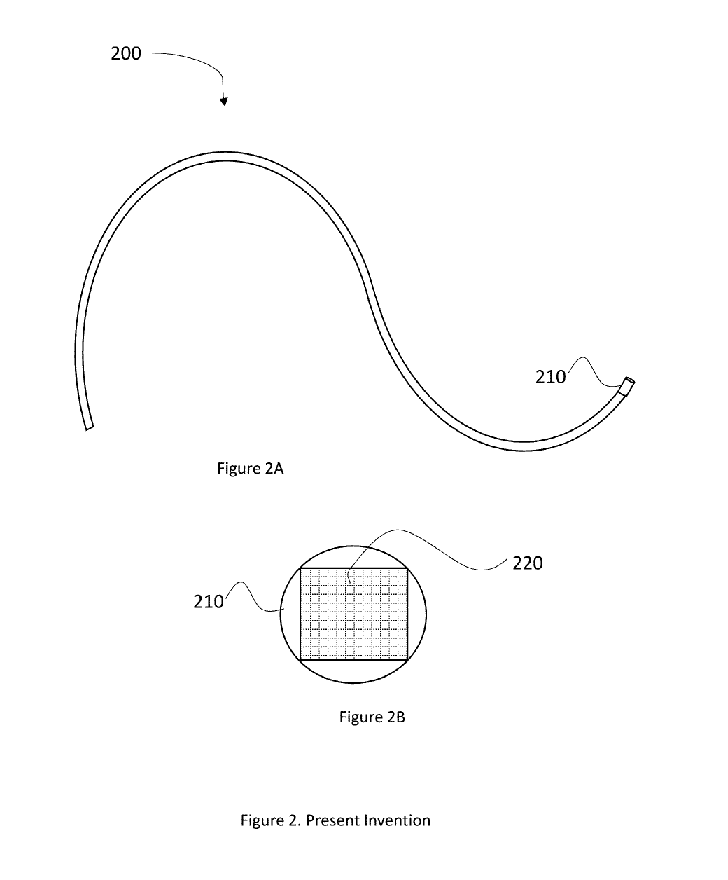

FIG. 2 illustrates details of the video/image camera system (200). In FIG. 2A, the video/image camera system (200) has an image sensor and lens (220) at the tip (210). The video/image camera system (200) is bendable, and the video/image camera system (200) has connecting cables to transmit image signals and power the image sensor (220). FIG. 2B is a top view of the video/image camera system (200) along the main axis of the video/image camera system (200). The image sensor and lens (220) is square-shaped, and the tip (210) is a circumcircle of the image sensor and lens (220) to maximum the area of the image sensor (220).

The image sensor and lens (220) can be powered by either a wirelessly rechargeable or a wirelessly non-rechargeable battery. For the rechargeable battery case, the battery can be recharged by placing a wireless charger (1600) close to the battery.

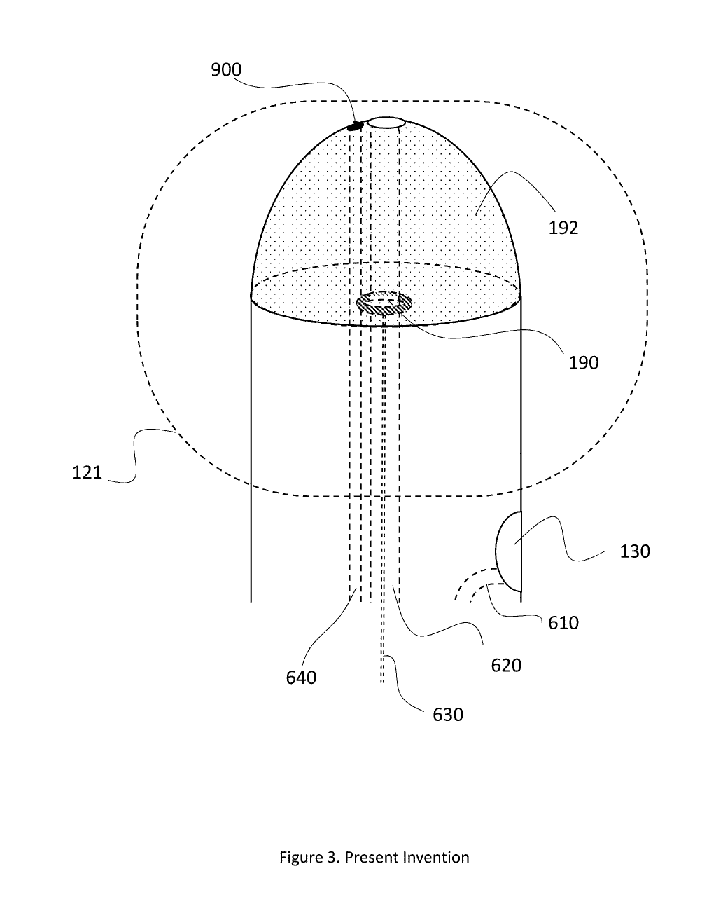

FIG. 3 illustrates the distal end (121) and its adjacent part. A transparent shell (192) is made of materials having properties of high light transmission, and it contains the lighting source (190) inside. Four lumens (610-640) are fabricated inside the generally tubular-shaped housing.

The transparent shell (192) has a waterproof design and is applied with a layer of hydrophobic coating on an outer surface to prevent blood or urine from remaining on the surface.

A first lumen (610) connects the drainage eye (130) to the first funnel (150). The first lumen (610) serves as a channel for urine to flow. The drainage eye (130) is apart from the distal end (121). The drainage eye (130) is an inlet for urine inside the patient's bladder.

A second lumen (620) connects the transparent shell (192) with the second funnel (140), providing a space for placing the video/image camera system (200). The tip (210) is placed against the front top of the catheter's distal end (121). The image sensor and lens (220) can capture images of the patient's bladder.

A third lumen (630) connects the lighting source (190) with the third funnel (160). Cables are placed inside the third lumen (630) and connected to the lighting source (190) for power transmission. The cables have terminals at the third funnel (160) and connect to an external power supply.

In the case that the lighting source (190) is wirelessly powered by an external power source, the third lumen (630) is not necessary. In the case that the lighting source (190) has a built-in battery, the battery can be either rechargeable or non-rechargeable. If the battery is rechargeable, it can be recharged by placing a wireless charger close to itself.

A fourth lumen (640) connects an opening (900) on the transparent shell (192) with the fourth funnel (170). The fourth funnel (170) serves as an irrigation port, an inlet for saline solution that is used to clean the outer surface of the transparent shell (192). Liquid medicine can also flow into the patient's bladder from the fourth funnel (170).

The fourth lumen (640) and the fourth funnel (170) are also for injecting other fluid, or gas such as CO₂ or air, or inserting laser/optical/medical treatment accessories.

The lighting source (510) is selected from LED, OLED, μLED, infrared device, laser device, and X-ray device depending on design requirements. The different lighting source emits lights of different wavelengths.

The lighting source (190) can be removed from the catheter for recycling.

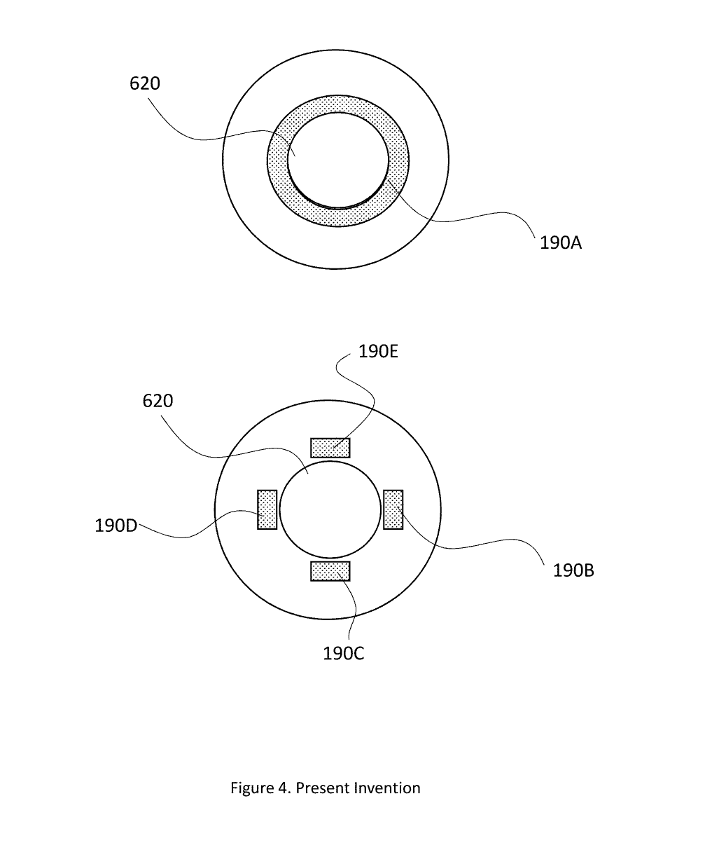

Two embodiments of the configuration of the third lumen (630) and the lighting source (190) are shown in FIG. 4. In the first embodiment, the lighting source (190A) is a ring-shaped lighting source surrounding the second lumen (620). In the second embodiment, the lighting sources (190B-190E) are rectangle-shaped lighting sources, arranged in a configuration surrounding the second lumen (620).

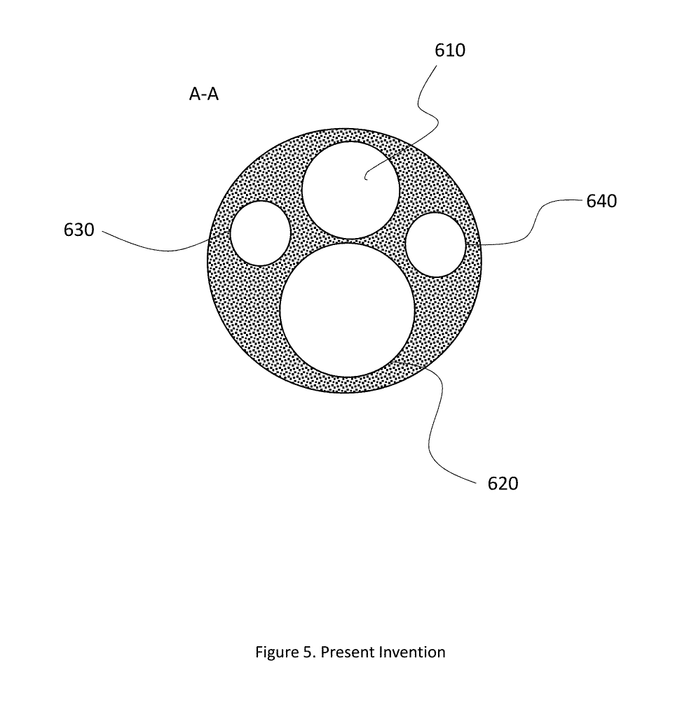

FIG. 5 illustrates a cross-section view of the catheter along a direction A-A referenced in FIG. 1. Four circular lumens (610-640) are fabricated inside the generally tubular-shaped housing.

While in use, the catheter is placed inside a patient's bladder. The lighting source (190) is on and it emits lights through the transparent shell (192). The image sensor and lens (220) on the tip (210) of the video/image camera system (200) captures images of the patient's bladder. After use, both the video/image camera system (200) and the lighting source (190) can be removed from the catheter.

The video/image camera system (200) and the lighting source (190) can be reused after proper sterilization procedures.

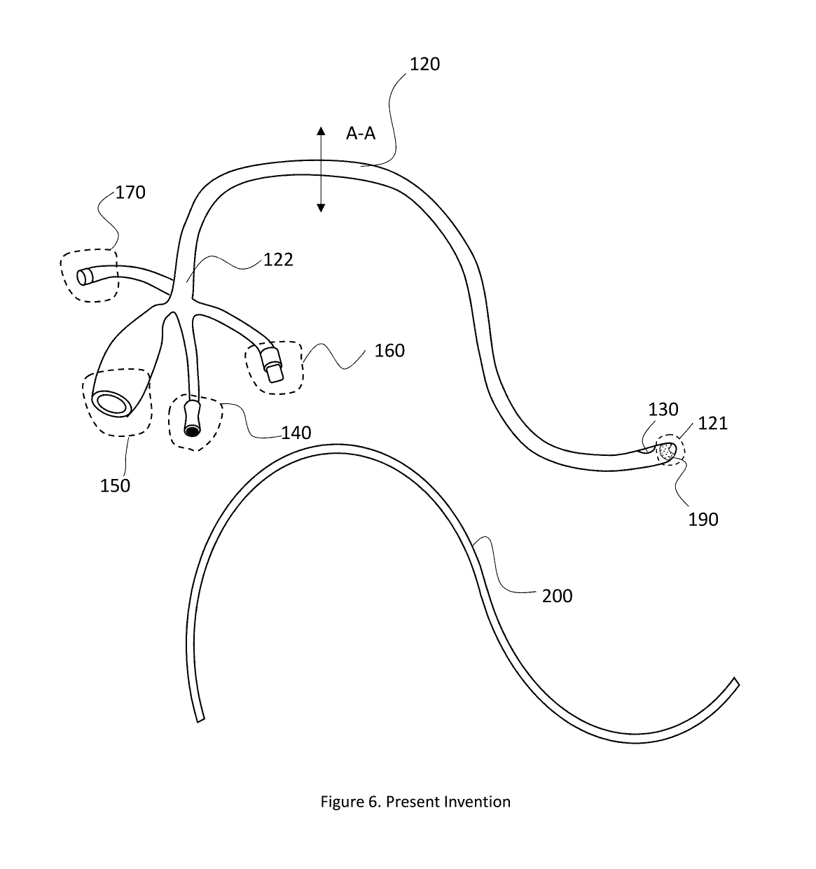

FIG. 6 illustrates the second embodiment of the present invention. In this embodiment, the present invention includes two parts including a catheter and a video/image camera system (200). The tubular-shaped catheter shaft (120) has one end connected to the distal end (121), and another end connected to the proximal end (122) where four funnels (140-170) joint. The lighting source (190) is installed inside the transparent shell (192) of the catheter. The catheter has a lumen reserved for inserting the video/image camera system (200).

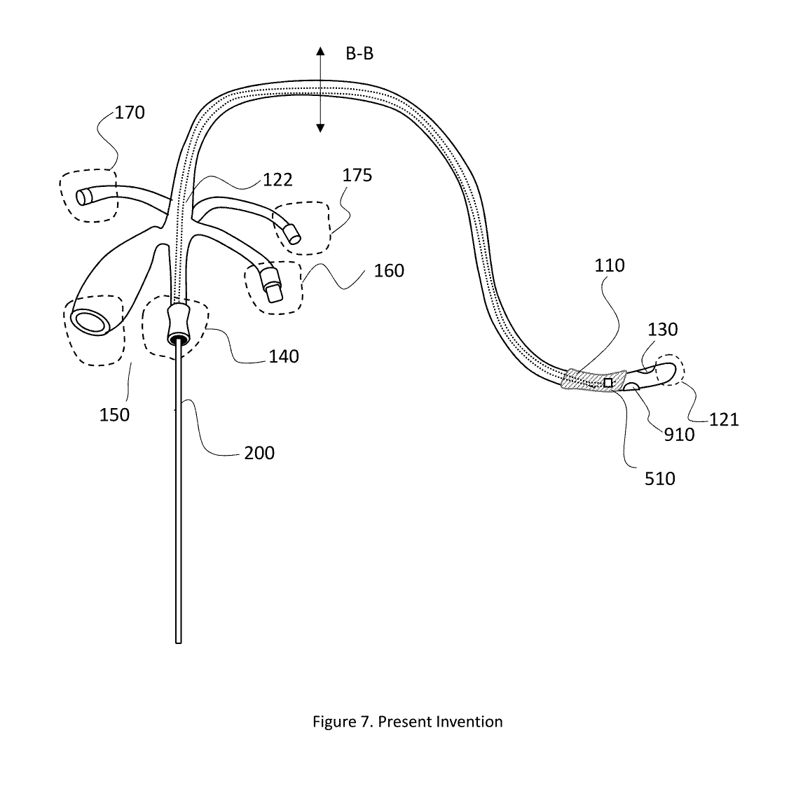

The third embodiment of the present invention is illustrated in FIG. 7. The catheter with a preinstalled video/image camera system (200) is generally tubular-shaped. The catheter shaft (120) has one end connected to the distal end (121), and another end connected to the proximal end (122) where five funnels (140-175) joint together. While in use, the distal end (121) of the catheter is inserted into a patient's bladder, and the balloon (110) is inflated by saline solution or air to prevent the catheter from sliding out from the patient's bladder neck. The image sensor and lens (220) of the video/image camera system (200) can be inserted into the balloon (110). The lighting source (510) can also be inserted into the balloon (110) for providing sufficient illuminations

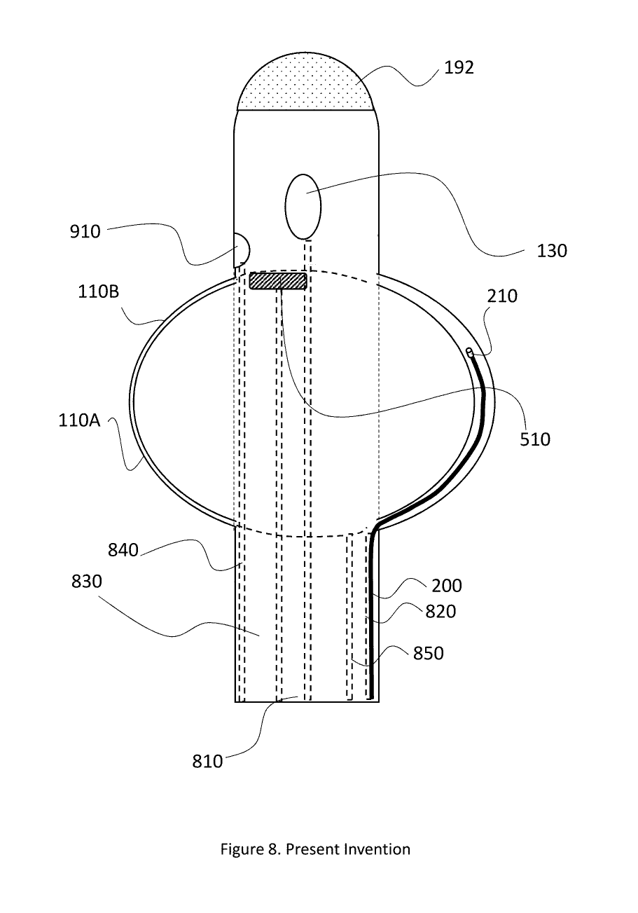

FIG. 8 illustrates a part of the catheter with an inflated balloon (110). The balloon (110) is made of a transparent material that can conduct lights. The balloon (110) has two layers, an outer layer (110A) and an inner layer (110B). The video/image camera system (200) can enter the interlayer sandwiched by the outer layer (110A) and the inner layer (110B). Saline solution or air is injected to the space surrounded by the inner layer (110B) to inflate the balloon (110). The interlayer of the balloon (110) is a part of the second lumen (820) allowing the passing of the video/image camera system (200). In FIG. 7, the lighting source (510) and the tip (210) of the video/image camera system (200) are placed at the balloon (110).

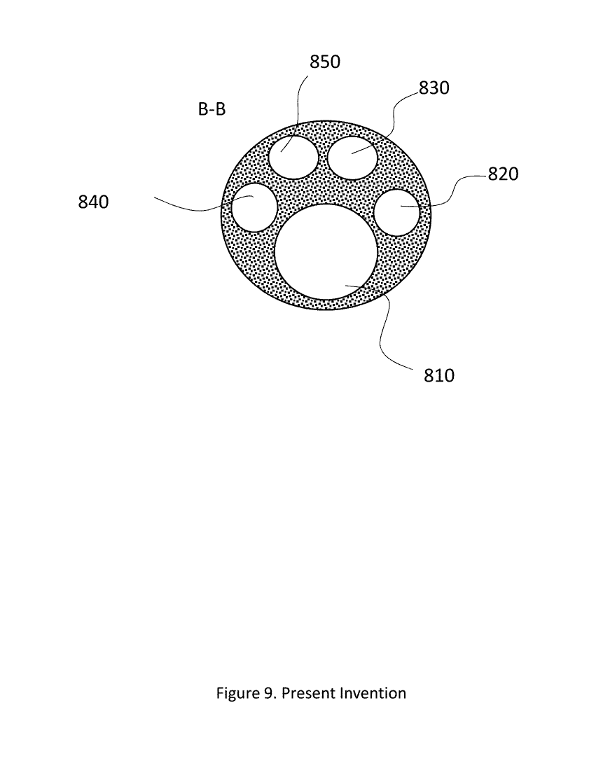

FIG. 9 illustrates a cross sectional view of the catheter along a direction B-B referenced in FIG. 7. The five lumens are fabricated inside the generally tubular-shaped housing. The lumens can be designed into different shapes according to design requirements.

The first lumen (810) connects the drainage eye (130) with a first funnel (150), used as a channel for urine flow. The second lumen (820) connects the transparent shell (192) with the interlayer space of the balloon (110) to the second funnel (140), allowing the video/image camera system (200) to pass through. The third lumen (830) connects the distal end (121) with the third funnel (160). A thin wire-bundle with suitable length and diameter is assembled inside the third lumen (830) to power the lighting source (510). A fourth lumen (840) connects the fluid opening (910) with the fourth funnel (175). The fourth funnel (175) is an inlet for the washing fluid. The fluid can wash and clean the blood or urine remains on the outer surface of the balloon (110). The fourth lumen (840) and fourth funnel (175) are also for injecting other fluid, or gas such as CO₂ or air, or inserting laser/optical/medical treatment accessories. The fifth funnel (170) is used as a bidirectional port for air or saline solution to inflate or deflate the balloon (110). The fifth lumen (850) connects the inner space of the balloon (110) to the fifth funnel (170).

The video/image camera system (200) enters the catheter through a different and isolated lumen from the one for urine drainage. Thus, the video/image camera system (200) is not contaminated by urine or blood.

The lighting source (510) is first placed at the distal end (121) together with the tip (210) of the video/image camera system (200). The lighting source (510) is behind the tip (210) relative to the upfront surface of the transparent shell (192).

The video/image camera system (200) can be pulled along the second lumen (820) to a position inside the balloon (110). The lighting source (510) can correspondingly be moved to a position around or inside the balloon (110) to provide sufficient illuminations for the video/image camera system (200).

In the case that the lighting source (510) is wirelessly powered by an external power source, the third lumen (830) is not necessary. In the case that the lighting source (510) has a built-in battery, the battery can be either rechargeable or non-rechargeable. If the battery is rechargeable, it can be recharged by placing a wireless charger close to itself.

The lighting source (510) is selected from LED, OLED, μLED, infrared device, laser device, and X-ray device depending on design requirements. The different lighting source emits lights of different wavelengths for various applications.

Two layers of the balloon (110) are made of transparent materials having elastic deformation capability. The balloon (110) has a sufficient distance apart from the drainage eye. Saline solution or air is injected to the fourth funnel (170) and the fifth lumen (850), and it then inflates the balloon (110). The inflated balloon (110) prevents the catheter from sliding out of the patient's bladder. The saline solution or air can be discharged to deflate the balloon (110) via the same path.

While in use, the catheter is inserted into the patient's bladder. The balloon (110) is inflated, and the lighting source (510) is on. If necessary, the lighting source (510) is pulled to the position of the transparent balloon (110) for illuminations. The image sensor and lens (220) of the video/image camera system (200) can be pulled into the interlayer of the balloon (110) for capturing images. In case blood or urine remains on the outer surface of the balloon (110) and it further blurs the images taken by the camera system (200), fluid can be injected and sprayed from the fluid opening (910) via the fifth funnel (175) to clean the surface. After use, the video/image camera system (200) can be removed from the catheter.

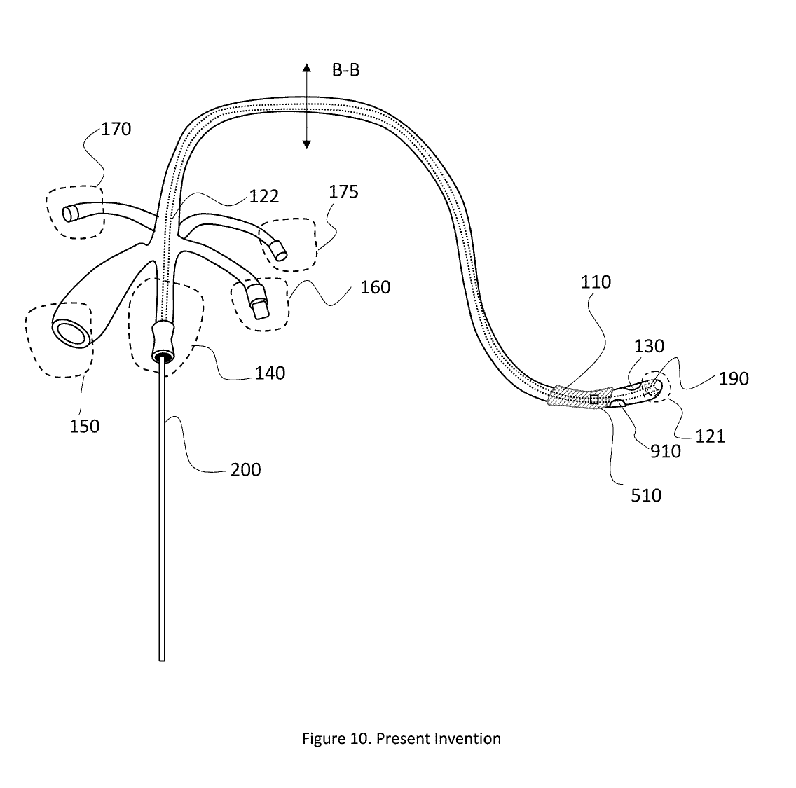

The fourth embodiment of the present invention is illustrated in FIG. 10. The catheter with a preinstalled video/image camera system (200) is generally tubular-shaped. The catheter shaft (120) has one end connected to the distal end (121), and another end connected to the proximal end (122) where five funnels (140-175) joint together. While in use, the distal end (121) of the catheter is inserted into a patient's bladder, and the balloon (110) is inflated by saline solution or air to prevent the catheter from sliding out from a patient's bladder neck. The video/image camera system (200) is placed in the second lumen (820), and the tip (210) including image sensor and lens is able to transmit images inside the patient's bladder. Two lighting source (190&510) provides sufficient illuminations for the image sensor.

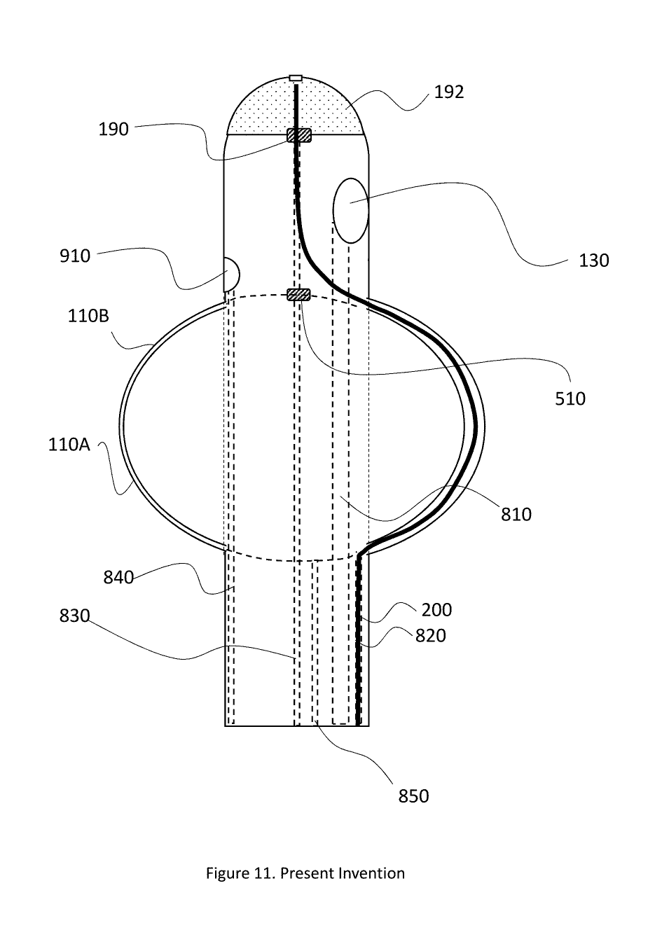

FIG. 11 illustrates a part of the catheter with an inflated balloon (110). The balloon is made of transparent materials having properties of high light transmission. The balloon (110) has two layers, an outer layer (110A) and an inner layer (110B). The video/image camera system (200) is able to enter the interlayer sandwiched by the outer layer (110A) and the inner layer (110B). Saline solution or air is injected into the space surround by the inner layer (110B) to inflate the balloon (110).

The first lumen (810) connects the drainage eye (130) with a first funnel (150) as a channel for urine flow. The second lumen (820) connects the transparent shell (192) with the interlayer space of the balloon (110) to the second funnel (140), and it allows the video/image camera system (200) to pass through. The third lumen (830) connects the distal end (121) with the third funnel (160). A thin wire bundle with suitable length and diameter is assembled inside the third lumen (830) for transmitting power to the lighting source (510). A fourth lumen (840) connects the fluid opening (910) with the fourth funnel (170) that is an inlet for the washing fluid. The fluid is sprayed on the outer surface of observation to clean the blood or urine remains as needed. The fourth lumen (840) and fourth funnel (170) are also for injecting other fluid, or gas such as CO₂, or medicine, or inserting laser/optical/medical treatment accessories. The fifth funnel (175) is used is a bidirectional port with air or saline solution to inflate or deflate the balloon (110). The fifth lumen (850) connects the inner space of the balloon (110) to the fifth funnel (175).

The video/image camera system (200) enters the catheter through a different and isolated lumen from the one for urine drainage. Thus, the video/image camera system (200) is not contaminated by urine or blood.

The lighting source (190) is installed at the catheter's distal end (121), and the lighting source (510) is installed at the portion of balloon (110). Two lighting sources are connected to the wires inside the third lumen (830). The on/off state of each lighting source can be controlled at the third funnel (160).

The lighting source (190) is selected from LED, OLED, μLED, infrared device, laser device, and X-ray device depending on design requirements. The different lighting source emits lights of different wavelengths for various applications.

In the case that the lighting source (190) is wirelessly powered by an external power source, the third lumen (830) is not necessary. In the case that the lighting source (190) has a built-in battery, the battery can be either rechargeable or non-rechargeable. If the battery is rechargeable, it can be recharged by placing a wireless charger close to itself.

The lighting source (510) is selected from LED, OLED, μLED, infrared device, laser device, and X-ray device depending on design requirements. The different lighting source emits lights of different wavelengths for various applications.

The lighting source (510) can be wirelessly powered by an external power source, the third lumen (830) is not necessary. In the case that the lighting source (510) has a built-in battery, the battery can be either rechargeable or non-rechargeable. If the battery is rechargeable, it can be recharged by placing a wireless charger close to itself.

Two layers of the balloon (110) are made of transparent materials having elastic deformation capability. The balloon (110) has a sufficient distance apart from the drainage eye. Saline solution or air is injected to the fourth funnel (170), and it then inflates the balloon (110). The inflated balloon (110) prevents the catheter from sliding out of the patient's bladder. The saline solution or air can be discharged to deflate the balloon (110) via the same path.

While in use, the catheter is inserted into the patient's bladder. The balloon (110) is inflated, and the lighting source (510) is on. If necessary, the lighting source (510) is pulled to the position of the transparent balloon (110) for illuminations. The video/image camera system (200) can be pulled into the interlayer of the balloon (110) for capturing images. In case blood or urine remains on the outer surface of the balloon (110) and it further blurs the images taken by the camera system (200), fluid can be injected and sprayed from the fluid opening (910) via the fifth funnel (175) to clean the surface. After use, the video/image camera system (200) can be removed from the catheter.

The present invention includes two embodiments of urinary catheters with endoscopic capabilities, and the video/image camera system is not built in. The urinary catheters in the fourth and the fifth embodiment have built-in lighting sources (190&510). The urinary catheters have specific lumens for the entrance of video/image camera systems (200). The video/image camera systems can be placed in either the catheter's distal end (121) or the balloon (110).

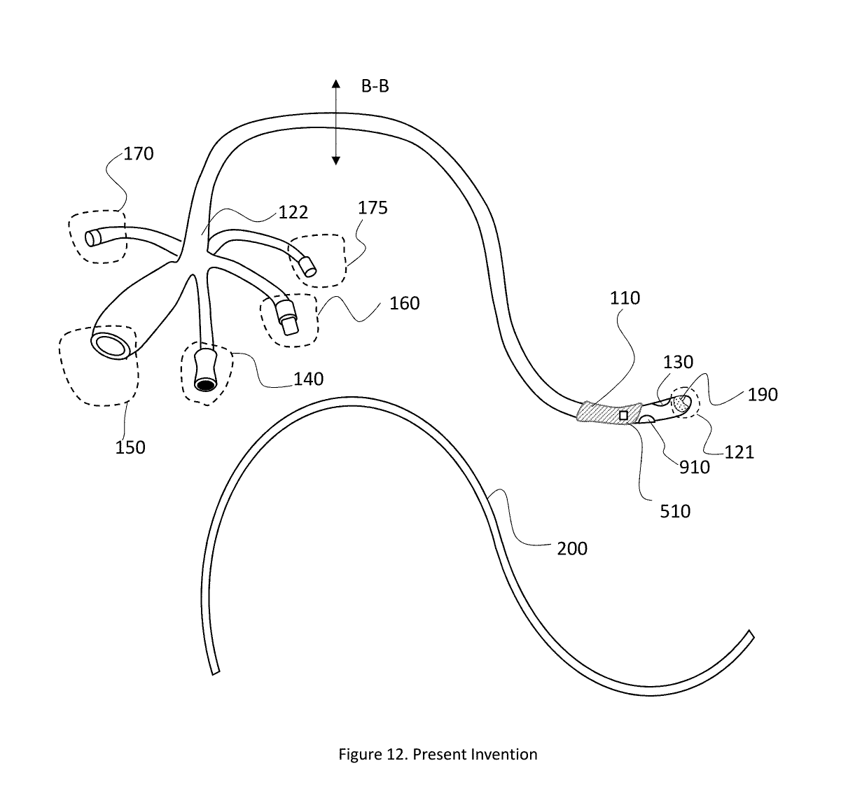

The fifth embodiment is shown in FIG. 12. The present invention includes a catheter and an independent video/image camera system (200). The catheter is generally tubular-shaped. The catheter shaft (120) has one end connected to the balloon and the distal end (121) of the catheter, and one proximal end (122) connected to four funnels. The catheter includes two built-in, non-removable lighting sources (190) and (510). The lighting source (190) is inside the transparent shell (192), and (510) is inside the balloon (110).

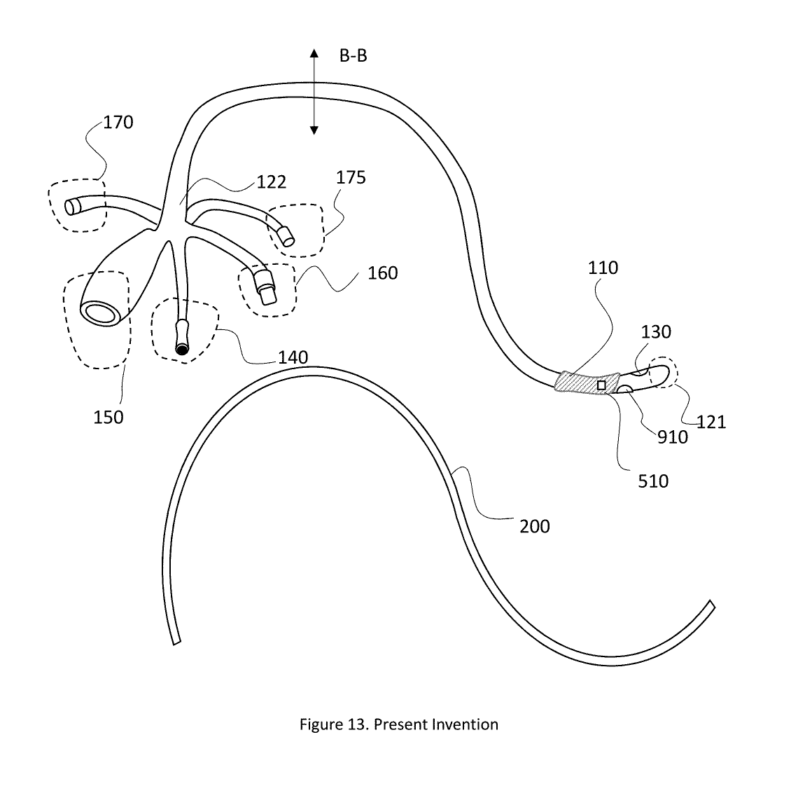

The sixth embodiment is shown in FIG. 13. The present invention includes a catheter and an independent video/image camera system (200). The catheter is generally tubular-shaped. The catheter shaft (120) has one end connected to the balloon and the distal end of the catheter, and another end connected to five funnels. A lighting source (190) is installed in the distal end (121) of the catheter.

In the seventh embodiment, the present invention includes two parts including a catheter and a video/image camera system (200). The tubular-shaped catheter shaft (120) has one end connected to the distal end (121), and another end connected to the proximal end (122) where four funnels (140-170) joint. The catheter has a balloon (110) used to prevent the catheter from sliding out of the bladder of the patient. The lighting source (190) is installed inside the transparent shell (192) of the catheter. The catheter has a lumen reserved for inserting the video/image camera system (200).

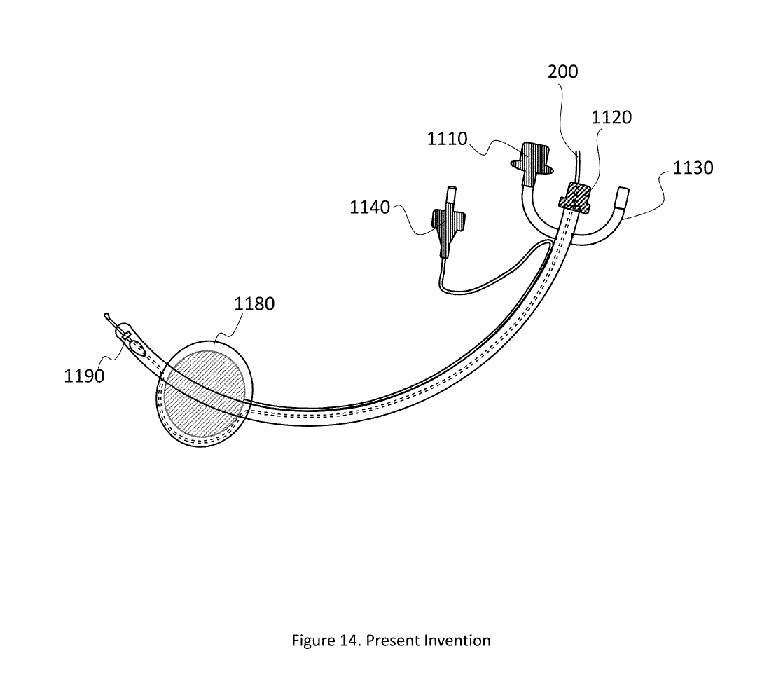

The eighth embodiment is illustrated in FIG. 14. The present invention includes a tracheal tube with a preinstalled video/image camera system (200). The tracheal tube has four funnels, a first funnel (1110), a second funnel (1120), a third funnel (1130), and a fourth funnel (1140). The fourth funnel (1140) connects to the balloon (1180) via the fourth lumen. A lighting source (1190) is installed at the distal end of the tracheal tube for providing illuminations for the video/image camera system (200). The balloon (1180) is double-layered, and the video/image camera system (200) can be placed in a lumen to enter the interlayer of the balloon (1180). The video/image camera system (200) can move freely in the lumen, and the video/image camera system (200) can be fully removed from the tracheal tube after use.

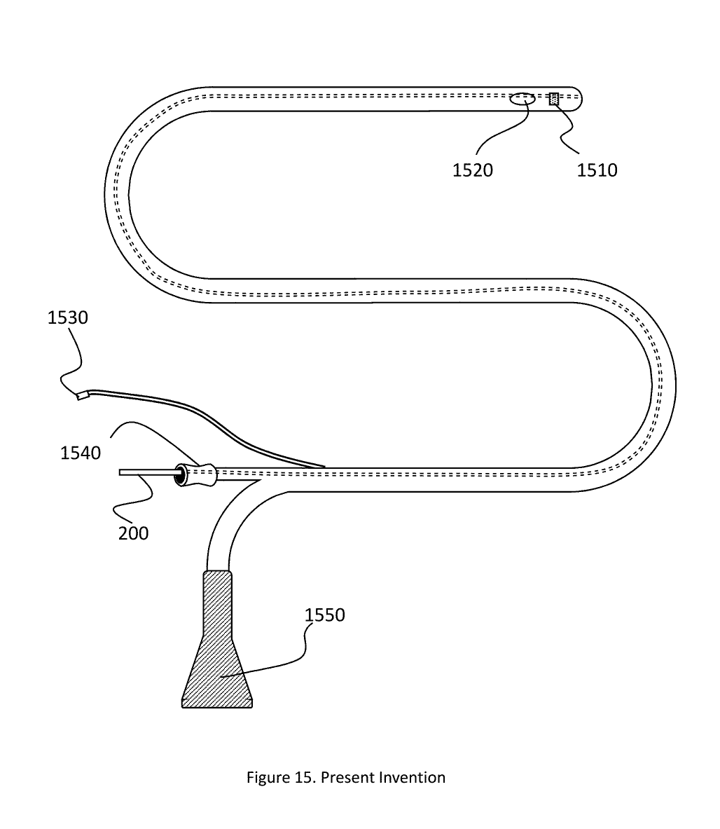

The ninth embodiment is illustrated in FIG. 15. The present invention includes a nasogastric tube with endoscopic capabilities with a preinstalled video/image camera system (200). The nasogastric tube has three funnels, a first funnel (1550), a second funnel (1540) and a third funnel (1530). The nasogastric as a lighting source (1510) installed inside a transparent shell (192) at the distal end of the nasogastric tube. The transparent shell (192) is made of materials having properties of high light transmission. The lighting source (1510) is connected to wires assembled through the third funnel (1530). The drainage eye (1520) is connected to the first funnel (1550). While in use, the lighting source (1510) is on, and it emits lights through the transparent shell (192) to provide illuminations for the video/image camera system (200).

The video/image camera system (200) does not consist of any attached/built-in lighting source. The camera system includes one or more image sensor(s), one or more lens(es), and cables/wires that electrically connect to the image sensor(s), and it can be powered by either an external power supply or an attached battery. The cable/wire bundle includes video signal cables, control signal cables, and a set of power supply wires.

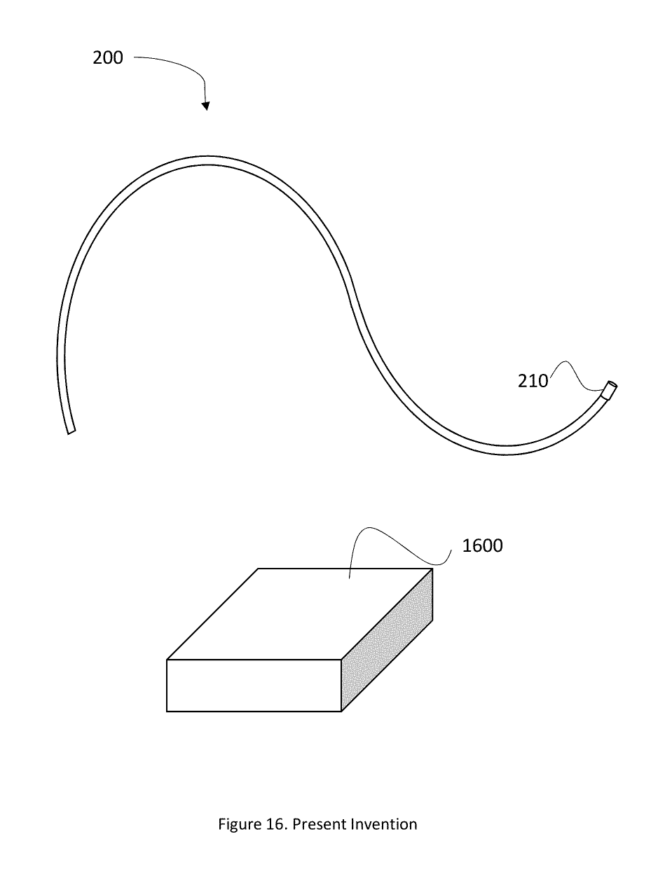

FIG. 16 illustrates an embodiment of the video/image camera that is powered by an attached battery. The battery can be either wirelessly rechargeable or non-rechargeable. An external wireless charger (1600) is needed if the battery is rechargeable. In this embodiment, the cable/wire bundle connecting to the image sensors doesn't need power supply wires.

The present invention includes a method of endoscopy by transforming medical tubes that have been widely applied in many medical areas. The independent video/image camera system (200) can be inserted into the medical tube with add-on endoscopic capabilities via the second lumen, and the medical tube together with the camera system is inserted into patient's body.

1. A medical tube with Add-on endoscopic capabilities (MTEC) comprising
a. a general or specialized medical tube or a kit of medical tubing (MT), chosen from a group of, including but not limited to, catheter, urinary catheter, gastric tube, nasogastric tube, feeding tube, rectal tube, and tracheal tube; and
b. one or more lighting source(s) on/in the MT.

2. The MTEC of claim 1 wherein the lighting source(s)
a. is/are located at a desired/needed location on/in the MT;
b. is/are powered by
i. at least one battery attached to the lighting source(s) wherein the battery is installed on the MT and is either wirelessly rechargeable or wired rechargeable or non-rechargeable; or
ii. at least one external power supply via a pair of electrical wires embedded in the MT;
c. comprise(s) one or more device(s) chosen from a group of LED, OLED, μLED, infrared device, laser device, and X-ray device; and
d. is/are made as either (a) built-in part(s) inside the MT or (a) movable/removable part(s).

3. The MTEC of claim 1 wherein the MT, having a generally tubular-shaped housing (GTSH), expressed in an X-Y-Z Cartesian coordinate system, with a tubular axis pointing along an Z-direction and a tubular cross section lying in a X-Y plane, comprises
a. a distal end (DE) comprising
i) a transparent shell with waterproof design (TSW); and
ii) an open end (OE);
b. a proximal end (PE) having a sufficient distance from the distal end (DE) at the −Z direction;
c. one or more drainage eye(s), near the distal end with a sufficient distance at the −Z direction from the distal end;
d. a first lumen between the proximal end (PE) and the drainage eye(s);
e. a first funnel connected to the first lumen at the proximal end (PE) and extending the first lumen;
f. a second lumen between the proximal end (PE) and the transparent shell with waterproof design (TSW) at the distal end (DE);
g. a second funnel connected to the second lumen at the proximal end and extending the second lumen;
h. a third lumen, if needed, between the proximal end (PE) and the transparent shell with waterproof design (TSW) at the distal end (DE);
i. a third funnel, if needed, connected to the third lumen at the proximal end and extending the third lumen;
j. a fourth lumen between the proximal end (PE) and the open end (OE) at the distal end (DE); and
k. a fourth funnel connected to the fourth lumen at the proximal end (PE) and extending the fourth lumen.

4. The MTEC of claim 3 wherein
a. the drainage eye(s), the first lumen, and the first funnel are for fluid drainage in either Z or −Z directions;
b. the second lumen comprises an endoscopic capability including a suitable shape and a sufficient size for at least one video/image camera without requiring any attached/built-in lighting source (NLVC) to be embedded in or inserted from the second funnel to a desired location along the MT including the transparent shell with waterproof design (TSW) at the distal end (DE) and pulled out from there to another location along the MT and even out from the MT, wherein the NLVC
i. does not require any light source attached/built to the VC(s);
ii. has/have an ability of taking videos/images towards a desired direction; and
iii. does/do not have to be installed or preinstalled until a time of a use of the MT together with the NLVC.
c. the third lumen and third funnel are filled with wires electrically connected the light source(s) for transmission if the lighting source(s) is/are powered by at least one external power;
d. the fourth lumen and fourth funnel are for water, or fluid, or Saline solution, or nutrients, or applicable gas(es), such as CO₂, or air, or medicine injection, or laser/optical/medical treatment accessories;
e. all the numbered lumens together with their connected funnels are isolated to each other; and
f. the transparent shell with waterproof design (TSW)
i. is for hosting the lighting source(s) and the video/image camera(s); and
ii. comprises a hydrophobic coating on an outer surface of the TSW.

5. The MTEC of claim 1 wherein the MT alternatively comprises
a. a distal end (DE) comprising
a) a transparent shell with waterproof design (TSW); and
b) an open end (OE);
b. one or more drainage eye(s), near the distal end with a sufficient distance at the −Z direction from the distal end, capable of accessing the first lumen;
c. a balloon comprising two layers of transparent materials wherein there is a lumen between the two layers of transparent materials, with a sufficient distance at the −Z direction from the drainage eye(s);
d. a proximal end having a sufficient distance from the distal end at the −Z direction;
e. a first lumen between the proximal end and the drainage eye(s);
f. a first funnel connected to the first lumen at the proximal end and extending the first lumen;
g. a second lumen between the proximal end (PE) and the transparent shell with waterproof design (TSW) at the distal end (DE) going through the lumen between the two layers of transparent materials of the balloon;
h. a second funnel connected to the second lumen at the proximal end and extending the second lumen;
i. a third lumen between the proximal end (PE) and the transparent shell with waterproof design (TSW) at the distal end (DE);
j. a third funnel connected to the third lumen at the proximal end and extending the third lumen;
k. a fourth lumen between the proximal end (PE) and the open end (OE) at the distal end (DE);
l. a fourth funnel connected to the fourth lumen at the proximal end (PE) and extending the fourth lumen;
m. an open end near an end of the balloon;
n. a fifth lumen between the proximal end and open end near an end of the balloon; and
o. a fifth funnel connected to the fifth lumen at the proximal end (PE) and extending the fifth lumen.

6. The MTEC of claim 5 wherein
a. the drainage eye(s), the first lumen, and the first funnel are for fluid drainage in either Z or −Z directions;
b. the second lumen comprises an endoscopic capability including a suitable shape and a sufficient size for at least one video/image camera without requiring any attached/built-in lighting source (NLVC) to be embedded in or inserted from the second funnel to a desired location along the MT including the transparent shell with waterproof design (TSW) at the distal end (DE) and pulled out from there to another location along the MT and even out from the MT, wherein the NLVC
i. does not require any light source attached/built to the VC(s);
ii. has/have an ability of taking videos/images towards a desired direction; and
iii. does/do not have to be installed or preinstalled until a time of a use of the MT together with the NLVC;
iv. the third lumen and third funnel are filled with wires electrically connected the light source(s) for transmission if the lighting source(s) is/are powered by at least one external power;
c. the third lumen and third funnel are filled with wires electrically connected the light source(s) for transmission if the lighting source(s) is/are powered by at least one external power;
d. the fourth lumen and fourth funnel are for water, or fluid, or Saline solution, or nutrients, or applicable gas(es), such as CO₂, or air, or medicine injection, or laser/optical/medical treatment accessories;
e. all the numbered lumens together with their connected funnels are isolated to each other;
f. the transparent shell with waterproof design (TSW)
i. is for hosting the lighting source(s) and the video/image camera(s); and
ii. comprises a hydrophobic coating on an outer surface of the TSW;
g. the balloon comprises a hydrophobic coating on a part of its outer surface that is helpful for the NLVC(s) to face out and observe targets; and
h. all materials between the balloon and the third lumen at a section where the balloon is located are transparent.

7. The MTEC of claim 6 wherein the lumen between the two layers of transparent materials of the balloon is for hosting the video/image camera(s) as needed or desired by pulling or inserting the wires/cables connected/attached to the video/image camera(s) through the second lumen and the second funnel.

8. The MTEC of claim 6 wherein
a. the NLVC(s) comprises one or more image sensor(s) and lens(es);
b. the second lumen and second funnel are filled with wires/cables electrically connected to the NLVC(s); and
c. the NLVC(s) together with its/their associated/connected wires/cables are made as movable parts for being moved from one location to another along the third lumen or even out of the MT.

9. The MTEC of claim 6 wherein the lighting source(s)
a. is/are located anywhere along the MT including in or around the DE and/or the balloon, as desired or as needed,
b. is/are powered by
i. at least one battery attached to the lighting source(s) wherein the battery is installed on the MT and is either wirelessly rechargeable or wired rechargeable or non-rechargeable; or
ii. at least one external power supply via a pair of electrical wires embedded in the MT;
c. comprise(s) one or more device(s) chosen from a group of LED, OLED, μLED, infrared device, laser device, and X-ray device; and
d. is/are made as either (a) built-in part(s) inside the MT or (a) movable/removable part(s).

10. A video/image camera without requiring any attached/built-in lighting source (NLVC), for endoscopy, comprising
a. at least one image sensor and at least one lens in front of the image sensor;
b. a cable, electrically/electronically connected to the image sensor from its backside, with a sufficient length and diameter/thickness and sufficient stiffness; and
c. a thin sheath completely covering the cable except its terminals.

11. The NLVC of claim 10 is powered by
a. at least one battery attached to the video/image camera(s) wherein the battery is installed on/in the MT and is either wirelessly rechargeable or wired rechargeable or non-rechargeable; or
b. at least one external power supply via a pair of electrical wires embedded in the cable.

12. The NLVC of claim 10 further comprises
a. a cable, electrically/electronically connected to the image sensor from its backside, with a sufficient length and diameter/thickness and sufficient stiffness wherein the cable comprises
i. a set of power supply wires for connecting the image sensor to one or more power supply;
ii. a set of video signal wires for transmitting video data out to an external video recorder;
iii. a set of control signal wires; and
b. a thin insulation sheath completely covering the cable except its terminals.

13. The NLVC of claim 10 alternatively comprises
a. a built-in battery directly attached to the image sensor and its associated circuitry for powering the NLVC wherein the built-in battery is either wirelessly rechargeable or wired rechargeable or non-rechargeable; and
b. a wireless transceiver for wirelessly communications between the NLVC and an external video recording and control device.

14. A method of an endoscopic diagnosis or treatment comprising steps as follows:
a. checking and determining if any camera has been installed in the MTEC;
b. if no camera has been preinstalled in the MTEC,
i. inserting an NLVC into an MTEC via a dedicated/second funnel and then a dedicated/second lumen of the MTEC, and then
ii. inserting the MTEC into a human organ for a diagnosis or a treatment; otherwise
c. if a camera has been preinstalled in the MTEC, inserting the MTEC into a human organ for a diagnosis or a treatment without going through step b(i).
